(19) United States
(12) Patent Application Publication
(10) Pub. No.: US 2003/0014001 A1
(43) Pub. Date: Jan. 16, 2003

(54) Ankle brace apparatus and method

(76) Inventor: Michael L. Martin, Murietta, CA (US)

Correspondence Address: MICHAEL BEST & FRIEDRICH, LLP, 100 E WISCONSIN AVENUE, MILWAUKEE, WI, 53202, US

(21) Appl. No.: 10/176,363

(22) Filed: Jun. 20, 2002

Related U.S. Application Data
(60) Provisional application No. 60/299,604, filed on Jun. 20, 2001.

(51) Int. Cl. A61F 5/00

(52) U.S. Cl. 602/27; 602/16; 602/12

(57) ABSTRACT

An ankle brace attachable to a patient including a foot plate, a medial upright, a lateral upright, and a shin guard. The shin guard is positionable between the patient's leg and the medial upright and the lateral upright. The ankle brace can also include one or more distal straps and/or one or more proximal straps. The shin guard is positionable between the patient's leg and the distal and/or proximal straps.

### FIELD OF THE INVENTION

This invention relates generally to orthotic braces, and more particularly to an apparatus and method for an ankle brace.

### BACKGROUND OF THE INVENTION

A person's ankle can move in four different ways, namely, dorsiflexion, plantar flexion, inversion, and eversion. Dorsiflexion is the flexing of the foot at the ankle in order to raise the toes. Plantar flexion is the flexing of the foot at the ankle in order to lower the toes. Dorsiflexion and plantar flexion occur normally during walking or running. Inversion is the tilting of the foot so that at least part of the sole faces medially or inward. Eversion is the tilting of the foot so that at least part of the sole faces laterally or outward. Inversion and eversion often occur during walking or running and are the frequent cause of ankle injury.

Conventional ankle braces allow some dorsiflexion and plantar flexion, while preventing inversion and eversion. However, many conventional ankle braces cannot stabilize the patient's ankle adequately enough for sports that require constant changes in direction while the person is running, such as soccer, basketball, volleyball, or football. Many conventional ankle braces do not allow normal (i.e., relatively free) dorsiflexion and plantar flexion, while substantially preventing inversion and eversion. Also, conventional ankle braces that provide a high level of support to prevent inversion and eversion generally are not easy to attach to the leg, are not comfortable to wear, and are not simple in construction. Moreover, conventional ankle braces that have foot plates often do not fit well in the patient's shoe and are not customized for the size of the patient's foot.

Conventional ankle braces do not provide any protection for the patient's shin area while the person is playing sports such as soccer. In order to protect his or her shin, the patient must purchase a separate shin guard device and attempt to attach the shin guard device to his or her ankle in the same area where the ankle brace must be attached. Conventional shin guards available for soccer are too large to fit within most ankle braces and are not designed to wrap around most ankle braces. Even if the shin guard can initially be attached to the patient's ankle along with the ankle brace, the shin guard will not be adequately secured to stay in the desired position on the patient's leg as the patient runs and changes direction. Soccer players also generally wear one or more layers of socks with their shin guards. However, wearing layers of socks, a shin guard, and a separate ankle brace is bulky and uncomfortable while playing soccer.

In light of the problems and limitations described above, a need exists for a method and apparatus for an ankle brace that allows normal dorsiflexion and plantar flexion while substantially preventing inversion and eversion. A need also exists for an ankle brace that is easy to attach to a person's ankle, that is comfortable to wear, and that is simple in construction. A need further exists for an ankle brace having a foot plate customized for the patient's foot and/or sized to fit in the patient's shoe. Finally, a need exists for an ankle brace that includes a shin guard designed to be worn with the ankle brace for use in playing sports such as soccer.

### BRIEF DESCRIPTION OF THE DRAWINGS

The present invention is further described with reference to the accompanying drawings which show preferred embodiments of the present invention. Although not specifically described herein, it should be noted that the invention as disclosed in the accompanying drawings is illustrated by way of example only. The various elements and combinations of elements described below and illustrated in the drawings can be arranged and organized differently to result in embodiments which are still within the spirit and scope of the present invention.

In the drawings, wherein like reference numerals indicate like parts:

FIG. 1 is a perspective view of an ankle brace according to a preferred embodiment of the invention;

FIG. 2 is a side elevational view of the ankle brace of FIG. 1; and

FIG. 3 is a perspective view of a shin guard for use with the ankle brace of FIG. 1.

### DETAILED DESCRIPTION OF THE PREFERRED EMBODIMENTS

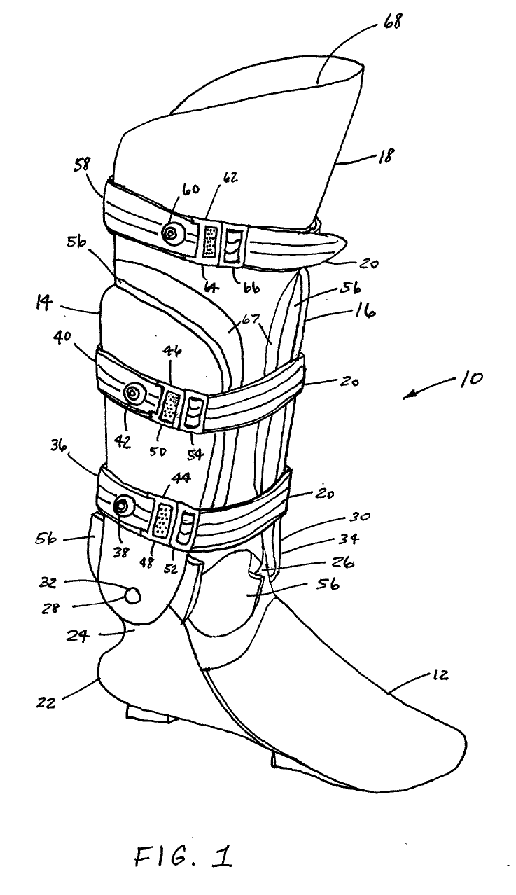

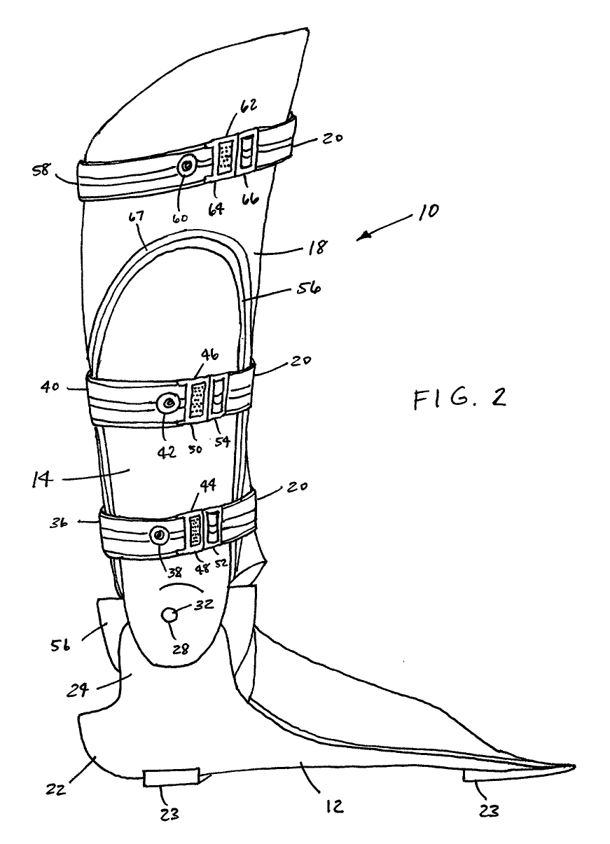

FIGS. 1-3 illustrate an ankle brace 10 embodying the invention. The ankle brace 10 illustrated in FIGS. 1-3 is designed for a patient's right foot. Although not specifically described herein, it should be understood that an ankle brace for use on a patient's left foot would be a mirror image of the ankle brace 10 for use on the patient's right foot. The ankle brace 10 includes a foot plate 12, a medial upright 14, a lateral upright 16, a shin guard 18, and one or more straps 20.

The foot plate 12 is preferably custom fabricated for each individual patient. Since the foot plate 12 is custom fabricated, the foot plate 12 can accommodate adults or children. However, the foot plate 12 can also be constructed according to one or more off-the-shelf sizes. For example, the foot plate 12 can be constructed according to typical shoe sizes.

To custom fabricate the foot plate 12, a practitioner (e.g., an orthotist fitting a patient for the ankle brace 10) preferably creates a negative cast of the patient's foot and lower leg using a kit provided by the manufacturer of the ankle brace 10. Before creating the negative cast, the practitioner marks on the patient's foot the borders of the medial and lateral malleoli, so that the marks will be transferred to the negative cast. The practitioner applies and secures with tape a protective cutting strip and a tube over the dorsal aspect of the patient's foot and ankle. Next, the practitioner applies a protective stockingette or baggy over the patient's entire foot and a portion of the patient's calf. The practitioner prepares a casting sock by submerging the casting sock in water. Once the casting sock is prepared, the practitioner positions the patient's foot in maximum dorsiflexion with subtalar neutral (e.g., the practitioner holds the talar joint in a non-weightbearing neutral position) and slides the casting sock onto the patient's foot and over the patient's heel and ankle. Working from the distal end of the patient's foot to the proximal portion of the patient's calf, the practitioner manipulates the casting sock by applying more water and using his or her hands to mold the contours of the patient's foot and to eliminate any wrinkles. The practitioner then allows the casting sock to set. After setting, the practitioner makes a horizontal V-shaped cut at the distal end of the tube and then removes the tube. With the tube removed, the practitioner can cut the casting sock along the channel created by the tube. The protective cutting strip protects the patient's shin and foot as the practitioner cuts the casting sock. Finally, the practitioner removes the casting sock from the patient's foot, secures the casting sock closed with one distal and one proximal rubber band, and sends the casting sock to the manufacturer. The interior of the casting sock is a negative mold of the patient's foot and lower leg.

Using the negative mold, the manufacture creates a positive mold of the patient's foot and lower calf in any suitable manner. The manufacture can make various biomechanical corrections to the positive mold. The manufacturer can specifically provide an arch support portion and a metatarsal support portion for the foot plate 12. The arch support portion can serve to locate the subtalar joint of the patient's foot in a neutral position and to aid in controlling subtalar motion. The arch support portion and the metatarsal support portion in combination can align the footpiece with respect to the patient's foot sole to help keep the ankle brace 10 in the proper anatomical position.

In order to create the custom foot plate 12, the manufacturer preferably vacuum forms a semi-rigid polypropylene around the foot bed portion of the positive cast of the patient's foot. The manufacturer also forms a heel cup 22 that is preferably integral with the foot plate 12. In some embodiments, for an ankle brace designed for adults, the heel cup 22 can be approximately 35 mm in height, and for an ankle brace designed for children, the heel cup 22 can be approximately 20 mm in height. The heel cup 22 can also be constructed of separate components that are coupled to the foot plate 12. Moreover, the heel cup 22 can be constructed of a different material than the foot plate 12. The foot plate 12 and the heel cup 22 can be constructed of various durable, impact resistant, resilient, low-maintenance materials (whether or not the materials can be custom formed to the positive case of the patient's foot). For example, the foot plate 12 and the heel cup 22 could also be constructed of polyethylene (e.g., UHMW Polyethylene or one of its commercial versions Lenite® by Westlake Plastics Company or Tivar 1000® by Poly Hi Solidue, Inc.), acetal polymer (e.g., DELRIN® by E. I. duPont de Numours and Company), ERTALYTE® (Quadrant DSM Polymer Corporation), Noryl® (General Electric Company), hardened polymers, metals, metal alloys, plastics, nylon, aluminum, fiberglass, carbon-fiber composite, etc.

The proximal side of the foot plate 12 (and in some embodiments at least part of the heel cup 22) are preferably lined with a metatarsal pad. The metatarsal pad is preferably constructed of a high-blown, closed-cell, chemically-crosslinked polyethylene foam, e.g., an ethylene vinyl acetate (EVA) foam. However, the metatarsal pad can be constructed of any other suitable material or combination of materials. The foot plate 12 can also include one or more stabilizing pads 23 coupled to the distal side of the foot plate 12. The stabilizing pads 23 are preferably constructed of a semi-rigid rubber material or any other suitable material. The stabilizing pads 23 can include an anterior toe pad positionable under the patient's toes that is flexible enough to allow the patient's toes to curl and move. The stabilizing pads 23 can also include a posterior heel pad positionable under the patient's heel.

The heel cup 22 includes a medial support 24 and a lateral support 26. The medial and lateral supports 24, 26 are preferably shaped to conform to the medial and lateral sides of the patient's ankle, including the patient's medial and lateral malleoli. Preferably, the medial support 24 is positioned more proximally than the lateral support 26. The medial support 24 includes a medial pivot hole 28 and the lateral support 26 includes a lateral pivot hole 30. From the negative cast of the patient's ankle, the manufacturer uses the marks indicating the borders of the medial and lateral malleoli to determine the axis of articulation for the patient's ankle. The manufacturer then aligns the medial and lateral pivot holes 28, 30 with the axis of articulation for the patient's ankle. Thus, the medial and lateral pivot holes 28, 30 are in line with the natural medial and lateral pivot points of the patient's ankle.

The medial and lateral uprights 14, 16 are preferably off-the-shelf components that are coupled to the medial and lateral supports 24, 26 of foot plate 12 once the manufacturer completes the custom foot plate 12. However, the medial and lateral uprights 14, 16 could also be custom fabricated from the negative mold of the patient's foot and calf. The medial and lateral uprights 14, 16 are preferably elongated, concave shells that generally conform to the anatomy of the patient's ankle and leg. More specifically, each one of the medial and lateral uprights 14, 16 preferably includes a first recess portion at its distal end to accommodate the malleolus and a second elongated recess portion to accommodate a portion of the side of the patient's calf.

The medial upright 14 is preferably pivotably attached to the foot plate 12 with a medial rotatable rivet 32 positioned through the medial pivot hole 28. Similarly, the lateral upright 16 is preferably pivotably attached to the foot plate 12 with a lateral rotatable rivet 34 positioned through the lateral pivot hole 30. Rather than rotatable rivets, the medial and lateral uprights 14, 16 can be pivotably attached to the foot plate 12 in any suitable manner, such as by using pivot pins, bolts and nuts, screws, or any other suitable fasteners. The rotatable rivets 32, 34 or other fasteners are preferably constructed of a strong, corrosion resistant, biocompatible material, such as aluminum, metals, metal alloys, etc.

The medial and lateral uprights 14, 16 are also preferably constructed of a semi-rigid, vacuum-formed polypropylene that is somewhat thinner than the polypropylene of the foot plate 12. However, the medial and lateral uprights 14, 16 can be constructed of other durable, impact resistant, resilient, low-maintenance materials, such as polyethylene (e.g., UHMW Polyethylene or one of its commercial versions Lenite® by Westlake Plastics Company or Tivar 1000® by Poly Hi Solidue, Inc.). The medial and lateral uprights 14, 16 can also be constructed of acetal polymer (e.g., DELRIN® by E. I. duPont de Numours and Company), ERTALYTE® (Quadrant DSM Polymer Corporation), Noryl® (General Electric Company), hardened polymers, steel and other metals, metal alloys, plastics, nylon, aluminum, fiberglass, carbon-fiber composite, etc.

The straps 20 preferably include a first distal strap 36, a second distal strap 40, and a proximal strap 58. The straps 20 are preferably constructed of C-FOLD® loop fastener material. A first end of the first distal strap 36 is preferably rigidly coupled to the medial upright 14 by a first rivet 38. A first end of the second distal strap 40 is preferably rigidly coupled to the medial upright 14 by a second rivet 42 above the first distal strap 36. Rather than rivets, the straps 36, 40 can also be coupled to the medial upright 14 using other suitable fasteners, such as bolts, screws, adhesives, hook and loop fastener, etc. The straps 36, 40 preferably extend around the circumference of the ankle brace 10 as created by the exterior portions of the medial and lateral uprights 14, 16. The straps 36, 40 preferably include brackets 44, 46, respectively, which are used to secure second ends of the straps 36, 40 once the straps 36, 40 are wrapped around the circumference of the ankle brace 10. More specifically, the brackets 44, 46 preferably include snap closures 48, 50. The female portions of the snap closures 48, 50 are preferably coupled to the first ends of the straps 36, 40 that are preferably rigidly coupled to the medial upright 14. The male portions of the snap closures 48, 50 are preferably coupled along the lengths of the straps 36, 40. The male portions of the snap closures 48, 50 preferably include double-square buckles 52, 54 through which the second ends of the straps 36, 40 are adjustably secured. The second ends of the straps 36, 40 are threaded around and through the buckles 52, 54.

The straps 36, 40 can be tightened by pulling the second ends of the straps 36, 40 away from the buckles 52, 54 (i. e., a double-compound pull is used to tighten the straps 36, 40). When the male portions of the snap closures 48, 50 are inserted into the female portions of the snap closures 48, 50, the ankle brace 10 can be tightened by pulling the second ends of the straps 36, 40. Thus, the patient can cinch the straps 36, 40 in order to reduce the circumference of the ankle brace 10 according to the fit he or she desires. Preferably, the same size medial and lateral uprights 14, 16 and straps 36, 40 can accommodate adults or children. In one embodiment, the circumference created by the medial and lateral uprights 14, 16 can initially be approximately 13 to 16½ inches and can be reduced to approximately 9 inches. The arrangement of the straps 36, 40 extending around the circumference of the medial and lateral uprights 14, 16 provides additional support to the patient's tibia. The patient can also loosen the straps 36, 40 by adjusting the straps 36, 40 with respect to the buckles 52, 54. The second ends of the straps 36, 40 preferably include sections of hook and loop fastener, so that the second ends of the straps 36, 40 can be releasably secured to the length of the straps 36, 40 positioned across the patient's shin. Rather than or in addition to the brackets 44, 46, the straps 36, 40 can be secured in the desired position in any suitable manner (e.g., with bolts, screws, buckles, clips, mating pins and apertures, rivets, threaded connections, snap-fit connections, press-fit connections, etc.).

Preferably, the medial and lateral uprights 14, 16 are at least partially lined with one or more liner pads 56. The liner pads 56 are preferably compression foam covered with fabric material. The liner pads 56 can also be constructed of a soft, thermo-absorbent material that provides a comfortable contact surface and conducts heat and/or moisture away from the patient's body. One such thermo-absorbent material is styrene butadiene foam (e.g., THERMASORB® by Frisby Technologies, Inc.). The liner pads 56 can also be constructed of one or more other suitable materials (whether capable of directing heat and/or moisture away from the patient's body or not), such as other closed-cell foams, urethane foams, soft plastics, or fabrics (e.g., cotton and cotton blends). The liner pads 56 preferably include an exterior side including hook and loop fastener and an interior side having a velveteen or deer skin texture that provides a comfortable contact surface with the patient's ankle and leg. The hook and loop fastener is used to couple the liner pads 56 to the medial and lateral uprights 14, 16. The liner pads 56 are preferably removable and their location on the uprights 14, 16 is adjustable according to the patient's needs. The liner pads 56 can also be connected to the medial and lateral uprights 14, 16 in any other suitable manner (whether or not they are removable or adjustable), such as with adhesives, elastic bands, snap-fit connections, buckles, clips, rivets, press-fit connections, by integrating the liner pads 56 with the medial and lateral uprights 14, 16, etc. Preferably, the liner pads 56 extend slightly beyond the perimeter of the medial and lateral uprights 14, 16 to prevent the perimeter of the medial and lateral uprights 14, 16 from digging into the patient's leg. Additional liner pads 56 can also be coupled to the medial and lateral uprights 14, 16, the shin guard 18, and/or the medial and lateral supports 24, 26 in order to provide cushioning between the patient's medial and lateral malleoli and the ankle brace 10.

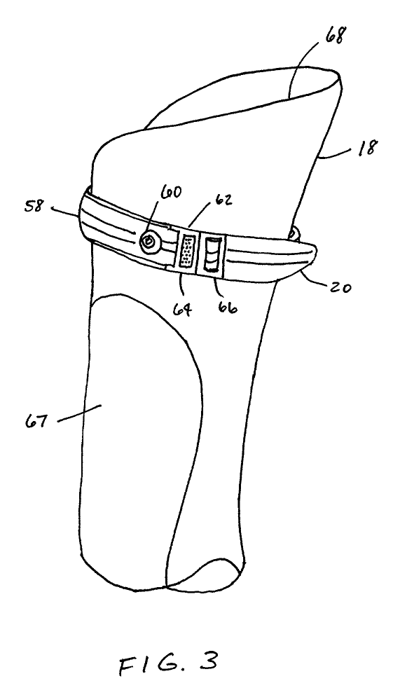

As shown in FIG. 1, the shin guard 18 of the ankle brace 10 is preferably positioned between the patient's shin and the medial and lateral uprights 14, 16 in a partially overlapping relationship with the medial and lateral uprights 14, 16. The shin guard 18 preferably covers the medial side, the shin area, and the lateral side of the patient's leg, but does not cover the entire posterior side of the patient's leg. The shin guard 18 overlaps with the medial upright 14 on the medial side of the patient's leg and overlaps with the lateral upright 16 on the lateral side of the patient's leg. In other embodiments, the shin guard 18 extends completely around the patient's leg or, conversely, only covers the shin area of the patient's leg. As shown in FIG. 3, the shin guard 18 is preferably substantially cylindrical in shape and has a larger circumference at its proximal end than its distal end.

Preferably, the shin guard 18 is at least partially secured between the patient's shin and the medial and lateral uprights 14, 16 by the first and second distal straps 36, 40. However, the shin guard 18 preferably also includes the proximal strap 58 in order to independently secure and conform the circumference of the shin guard 18 to the patient's calf. Similar to the straps 36, 40, a first end of the proximal strap 58 is rigidly coupled to the shin guard 18 by a third rivet 60. Rather than a rivet, the strap 58 can also be coupled to the shin guard 18 using other suitable fasteners, such as bolts, screws, adhesives, hook and loop fastener, etc. The strap 58 preferably extends around the circumference of the shin guard 18 and the patient's calf. The strap 58 preferably includes a bracket 62 to secure the second end of the strap 58 once the strap 58 is wrapped around the shin guard 18 and the patient's calf. More specifically, the bracket 62 includes a snap closure 64. The female portion of the snap closure 64 is coupled to the first end of the strap 58 that is rigidly coupled to the shin guard 18. The male portion of the snap closure 64 is coupled along the length of the strap 58. The male portion of the snap closure 64 includes a double-square buckle 66 through which the second end of the strap 58 is attached. The second end of the strap 58 is threaded around and through the buckle 66.

The strap 58 can be tightened by pulling the second end of the strap 58 away from the buckle 66 (i.e., a double compound pull is used to tighten the strap 58). When the male portion is inserted into the female portion of the snap closure 64, the shin guard can be tightened by pulling the second end of the strap 58 in order to reduce the circumference of the shin guard 18 to conform to the patient's calf. Thus, the patient can cinch the strap 58 in order to tighten the shin guard 18 according to the fit he or she desires. In one embodiment, the circumference created by the shin guard 18 and the patient's calf can initially be approximately 17 inches and can be reduced to approximately 12 inches. The arrangement of the strap 58 around the circumference of patient's calf and the shin guard 18 provides additional support to the patient's tibia. The patient can also loosen the strap 58 by adjusting the strap 58 with respect to the buckle 66. The second end of the strap 58 also preferably includes hook and loop fastener, so that the second end of the strap 58 can be releasably secured to the length of the strap 58 positioned across the patient's shin. Rather than or in addition to the bracket 62, the strap 58 can be secured in the desired position in any suitable manner (e.g., with bolts, screws, buckles, clips, mating pins and apertures, rivets, threaded connections, snap-fit connections, press-fit connections, etc.).

The shin guard 18 is preferably constructed of a semi-rigid acrylic/poly vinyl carbon (PVC) alloy (e.g., KYDEX® by Kleerdex Company or Concealex by Blade-Tech). Preferably, one or more portions of the shin guard 18 can be cut off in order to reduce the length of the shin guard 18 according to the length of the patient's shin. For example, the shin guard 18 can be manufactured in a single size, and portions of the shin guard 18 can be cut to accommodate an adult or a child's shin length. The shin guard 18 can also be constructed of other durable, impact resistant, resilient, low-maintenance materials (whether or not the materials can be readily cut). For example, the shin guard 18 can be constructed of polyethylene (e.g., UHMW Polyethylene or one of its commercial versions Lenite® by Westlake Plastics Company or Tivar 1000® by Poly Hi Solidue, Inc.). The shin guard 18 can also be constructed of acetal polymer (e.g., DELRIN® by E. I. duPont de Numours and Company), ERTALYTE® (Quadrant DSM Polymer Corporation), Noryl® (General Electric Company), hardened polymers, metals, metal alloys, plastics, nylon, aluminum, fiberglass, carbon-fiber composite, etc.

Preferably, the shin guard 18 includes an interior side having a smooth surface and an exterior side having a textured surface. In addition, the exterior side of the shin guard 18 preferably includes relatively large sections 67 of hook and loop fastener in order to releasably secure the shin guard 18 to hook and loop fastener on the medial and lateral uprights 14, 16. The shin guard 18 can be removably and adjustably attached to the medial and lateral uprights 14, 16 when liner pads 56 are not attached to the medial and lateral uprights 14, 16. The shin guard 18 can be adjusted so that it is more distal or more proximal with respect to the medial and lateral uprights 14, 16 and can be secured in the desired distal/proximal position with the hook and loop fastener. When the shin guard 18 is attached, a medial malleolus pad 70 and a lateral malleolus pad 72 can be coupled to the shin guard 18 to cover the medial support 24 and the lateral support 26, respectively. The locations of medial and lateral malleolus pads 70, 72 can be adjusted according to the patient's needs. The shin guard 18, its proximal strap 58, and the medial and lateral malleolus pads 70, 72 can be removed completely from the ankle brace 10. This feature allows the patient to use the shin guard 18 while playing sports and then remove the shin guard 18 when it is no longer needed. After the shin guard 18 is removed, liner pads 56 can be placed on the medial and lateral uprights 14, 16.

The interior side of the shin guard 18 is also preferably lined with one or more liner pads 68. The liner pads 68 are also preferably a compression foam covered with a fabric material. The liner pads 68 can also be constructed of a soft, thermo-absorbent material that provides a comfortable contact surface and conducts heat and/or moisture away from the patient's body. One such type of material is a styrene butadiene foam (e.g., THERMASORB® by Frisby Technologies, Inc.). The liner pads 68 can also be constructed of one or more other suitable materials (whether capable of directing heat and/or moisture away from the patient's body or not), such as other closed-cell foams, urethane foams, soft plastics, or fabrics (e.g., cotton and cotton blends). The liner pads 68 preferably include an exterior side having hook and loop fastener and an interior side having a velveteen or deer skin texture that provides a comfortable contact surface with the patient's leg. The hook and loop fastener is used to couple the liner pads 68 to the interior of shin guard 18. The liner pads 68 are adjustable with respect to the shin guard 18 according to the patient's needs. The liner pads 68 can be connected to the shin guard 18 in any other suitable manner, such as with adhesives, elastic bands, snap-fit connections, buckles, clips, rivets, press-fit connections, by integrating the liner pads 68 with the shin guard 18, etc. In some embodiments, the liner pads 68 extend slightly beyond the perimeter of the shin guard 18 to prevent the perimeter of the shin guard 18 from digging into the patient's leg.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

We claim:

1. An ankle brace for attachment to a patient, the ankle brace comprising:
a foot plate;
a medial upright coupled to the foot plate;
a lateral upright coupled to the foot plate; and
a shin guard coupled to at least one of the medial upright and the lateral upright and positionable between the patient's leg and the medial upright and the lateral upright.

2. The ankle brace of claim 1, wherein the foot plate, the medial upright, and the lateral upright are constructed of a semi-rigid material.

3. The ankle brace of claim 2, wherein the semi-rigid material is a polypropylene.

4. The ankle brace of claim 1, and further comprising at least one distal strap coupled to at least one of the medial upright and the lateral upright.

5. The ankle brace of claim 4, wherein the at least one distal strap is positionable around a first circumference defined by the medial upright and the lateral upright, and wherein the shin guard is positionable between the patient's leg and the at least one distal strap.

6. The ankle brace of claim 5, wherein the at least one distal strap is an adjustable strap for use in increasing or decreasing the first circumference.

7. The ankle brace of claim 1, and further comprising at least one proximal strap coupled to the shin guard and positionable around a second circumference defined by the shin guard.

8. The ankle brace of claim 7, wherein the at least one proximal strap is an adjustable strap for use in increasing or decreasing the second circumference.

9. The ankle brace of claim 1, wherein the medial upright and the lateral upright are pivotably coupled to the foot plate.

10. The ankle brace of claim 1, wherein the foot plate extends substantially the entire length of the patient's foot.

11. The ankle brace of claim 1, wherein the foot plate is a custom-fabricated foot plate.

12. The ankle brace of claim 1, and farther comprising a heel cup coupled to the foot plate, a medial support rigidly coupled to the foot plate and pivotably coupled to the medial upright, and a lateral support rigidly coupled to the foot plate and pivotably coupled to the lateral upright.

13. The ankle brace of claim 1, wherein the shin guard is removably coupled to at least one of the medial upright and the lateral upright.

14. The ankle brace of claim 1, wherein the shin guard is constructed of an acrylic/poly vinyl carbon alloy.

15. The ankle brace of claim 1, wherein at least one liner pad at least partially constructed of compression foam is removably and adjustably coupled to at least one of the medial upright, the lateral upright, and the shin guard.

16. The ankle brace of claim 1, wherein an ethylene vinyl acetate foam pad is coupled to the foot plate.

17. An ankle brace for attachment to a patient, the ankle brace comprising;
a semi-rigid foot plate;
a semi-rigid medial upright pivotably coupled to the foot plate and positionable on a medial side of the patient's leg;
a semi-rigid lateral upright pivotably coupled to the foot plate and positionable on a lateral side of the patient's leg;
at least one distal strap coupled to at least one of the medial upright and the lateral upright and positionable around a first circumference defined by the medial upright and the lateral upright;
a shin guard coupled to at least one of the medial upright and the lateral upright and positionable between the patient's leg and the medial upright, the lateral upright, and the at least one distal strap; and
at least one proximal strap coupled to the shin guard and positionable around a second circumference defined by the shin guard.

18. The ankle brace of claim 17, wherein the foot plate, the medial upright, and the lateral upright are constructed of a polypropylene material.

19. The ankle brace of claim 17, wherein the at least one distal strap is an adjustable strap for use in increasing or decreasing the first circumference.

20. The ankle brace of claim 17, wherein the at least one proximal strap is an adjustable strap for use in increasing or decreasing the second circumference.

21. The ankle brace of claim 17, wherein the foot plate extends substantially the entire length of the patient's foot.

22. The ankle brace of claim 21, wherein the foot plate is a custom-fabricated foot plate.

23. The ankle brace of claim 17, and further comprising a heel cup coupled to the foot plate, a medial support rigidly coupled to the foot plate and pivotably coupled to the medial upright, and a lateral support rigidly coupled to the foot plate and pivotably coupled to the lateral upright.

24. The ankle brace of claim 17, wherein the shin guard is removably coupled to at least one of the medial upright and the lateral upright.

25. The ankle brace of claim 17, wherein the shin guard is constructed of an acrylic/poly vinyl carbon alloy.

26. The ankle brace of claim 17, wherein at least one liner pad at least partially constructed of compression foam is removably and adjustably coupled to at least one of the medial upright, the lateral upright, and the shin guard.

27. The ankle brace of claim 17, wherein an ethylene vinyl acetate foam pad is coupled to the foot plate.

28. A method of bracing a patient's ankle and protecting a patient's shin, the method comprising:
fabricating a custom foot plate;
attaching a medial upright and a lateral upright to the custom foot plate;
providing a shin guard for placement between the patient's leg and the medial upright and the lateral upright; and
cutting off a portion of the shin guard according to the length of the patient's shin.

29. The method of claim 28, and further comprising providing at least one distal strap coupled to at least one of the medial upright and the lateral upright and positionable around a first circumference defined by the medial upright and the lateral upright, and adjusting the at least one distal strap to increase or decrease the first circumference.

30. The method of claim 28, and further comprising providing at least one proximal strap coupled to the shin guard and positionable around a second circumference defined by the shin guard, and adjusting the at least one proximal strap to increase or decrease the second circumference.

31. The method of claim 28, and further comprising providing at least one adjustable liner pad coupleable to at least one of the medial upright, the lateral upright, and the shin guard.
